(19) United States
(12) Patent Application Publication
(10) Pub. No.: US 2010/0144515 A1
(43) Pub. Date: Jun. 10, 2010

(54) FLUORINATED DIALKOXY-DIIMINO CATALYST COMPONENTS

(75) Inventors: Jean-Francois Carpentier, Acigne (FR); Evgueni Kirillov, Rennes (FR); Thomas Christophe, Rance (FR); Abbas Razavi, Mons (BE)

Correspondence Address: FINA TECHNOLOGY INC, PO BOX 674412, HOUSTON, TX, 77267-4412, US

(73) Assignee: TOTAL PETROCHEMICALS RESEARCH FELUY, SENEFFE (BE)

(21) Appl. No.: 12/440,873

(22) PCT Filed: Sep. 14, 2007
(86) PCT No.: PCT/EP2007/059728

§ 371 Date: Jan. 25, 2010

(30) Foreign Application Priority Data
Sep. 15, 2006 (EP) 06120744.5

(51) Int. Cl. C08F 4/76; C07C 251/08; C07F 7/00; B01J 31/18

(52) U.S. Cl. 502/103; 564/278; 556/32; 502/152

(57) ABSTRACT

This invention relates to the fluorinated dialkoxy-diimine metal complexes and their use in catalyst system for the polymerisation or oligomerisation of ethylene and alpha-olefins.

The present invention discloses catalyst components based on fluorinated dialkoxy-diimino ligands, their method of preparation and their use in the polymerisation or oligomerisation of olefins.

Investigations to replace ubiquitous cyclopentadienyl-type ligands in modern coordination chemistry of early transition metals have become very popular in recent years. Hard, electronegative π-donor ligands such as aryloxides/alkoxides are attractive because they offer strong metal-oxygen bonds that are expected to stabilize complexes of these electropositive metals. Also, the great variety of these ligands conveniently obtained from alcohols allows considerable stereo-electronic variations. Accordingly, group 4 metal complexes of the type [MX₂(OZ)₂] and [MX₂(OZZO)], where OZ⁻ and OZZO²⁻ are monoanionic and chelating dianionic ligands respectively, have retained considerable attention. Most successful developments in terms of synthetic organometallic chemistry and catalysis have appeared using aryloxide (phenolate) ligands, with significant contributions of the groups of Fujita such as for example Matsui et al. (S. Matsui, M. Mitani, J. Saito, Y. Tohi, H. Makio, N. Matsukawa, Y. Takagi, K. Tsuru, M. Nitabaru, T. Nakano, H. Tanaka, N. Kashiwa, T. Fujita, in J. Am. Chem. Soc. 2001, 123, 6847-6856) or Kojoh et al. (S. Kojoh, T. Matsugi, J. Saito, M. Mitani, T. Fujita, N. Kashiwa, Chem. Lett. 2001, 822-823) or Saito et al. (J. Saito, M. Mitani, S. Matsui, J. Y. Mohri, S. Kojoh, N. Kashiwa, T. Fujita, Angew. Chem., Int. Ed. 2001, 40, 2918-2920; Angew. Chem. 2001, 113, 3002-3004) or Mitani et al. (M. Mitani, J. Mohri, Y. Yoshida, J. Saito, S. Ishii, K. Tsuru, S. Matsui, R. Furuyama, T. Nakano, H. Tanaka, S.-I. Kojoh, T. Matsugi, N. Kashiwa, T. Fujita, J. Am. Chem. Soc. 2002, 124, 3327-3336) or Saito et al. (J. Saito, M. Mitani, J. Mohri, S. Ishii, Y. Yoshida, T. Matsugi, S. Kojoh, N. Kashiwa, T. Fujita, T. Chem. Lett. 2001, 576-577) or Mitani et al. (M. Mitani, R. Furuyama, J.-I. Mohri, J. Saito, S. Ishii, H. Terao, N. Kashiwa, T. Fujita, J. Am. Chem. Soc. 2002, 124, 7888-7889) or of the group of Coates such as for example Tian and Coates (J. Tian, G. W. Coates, Angew. Chem., Int. Ed. 2000, 39, 3626-3629; Angew. Chem. 2000, 112, 3772-3775 or Tian et al, (J. Tian, P. D. Hustad, G. W. Coates, J. Am. Chem. Soc. 2001, 123, 5134-5135) or Mason and Coates (A. F. Mason, G. W. Coates, J. Am. Chem. Soc. 2004, 126, 16326-16327).

Comparatively, group 4 metal [MX₂(OZ)₂] and [MX₂(OZZO)] type complexes that incorporate simple (amino-)alkoxide ligands have been much less studied. This is mainly due to aggregation problems traditionally encountered with these relatively more basic ligands (as compared to aryloxides). A valuable approach to overcome this issue consists in introducing electron-withdrawing CF₃ groups a to the alkoxides; this generates increased intra- and inter-molecular repulsions and a much less basic alkoxide O-atom, and in turn a much reduced bridging ability. On this principle, Jordan et al. have developed “fluorous” group 4 complexes with two pyridine-alkoxide ligands such as for example in Tsukahara et at (T. Tsukahara, D. C. Swenson, R. F. Jordan, Organometallics 1997, 16, 3303-3313). Tetradentate diamino-dialkoxide {ONⁿNO}²⁻ ligands with a ethylene- (n=2), propylene- (n=3) or chiral 1,2-cyclohexyldiamine backbone flanked by two fluorinated tertiary alkoxides were recently prepared as well as neutral groups 3, 4 and 13 metal complexes derived thereof. They are described for example in Lavanant et al. (L. Lavanant, T.-Y. Chou, Y. Chi, C. W. Lehmann, L. Toupet, J.-F. Carpentier, Organometallics 2004, 23, 5450-5458) or in Amgoune et al. (A. Amgoune, L. Lavanant, C. M. Thomas, Y. Chi, R. Welter, S. Dagorne, J.-F. Carpentier, Organometallics, 2005, 24, 6279-6282) or in Kirillov et al. (E. Kirillov, L. Lavanant, C. M. Thomas, T. Roisnel, Y. Chi, J.-F. Carpentier, Chem. Eur. J. 2006, in press).

There is a demand for new catalyst systems having good activity and able to produce polymers or oligomers tailored to specific needs.

It is an aim of the present invention to prepare new catalyst components that can be used in the polymerisation or oligomerisation of olefins.

It is also an aim of the present invention to provide very active catalyst components.

It is another aim of the present invention to provide a method for homo- or co-polymerising or oligomerising olefins.

The present invention reaches, at least partially, any one of those aims.

Accordingly, the present invention discloses a class of fluorinated dialkoxy-diimine pro-ligands of formula

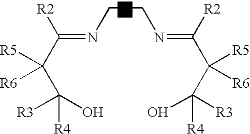

wherein

R¹ is a bridge having from 1 to 6 carbon atoms, substituted or unsubstituted and wherein the substituents if present may form a ring either with each other or with R²;

R² are each independently selected from hydrogen, aryl or alkyl having from 1 to 20 carbon atoms, optionally substituted with one or more fluorinated groups;

R³ and R⁴ are each independently selected from perfluorinated alkyl groups, from aryl or alkyl having from 1 to 20 carbon atoms, with the restriction that at least one of

R³ and R⁴ is a perfluorinated alkyl group;

R⁵ and R⁶ are each independently selected from hydrogen, alkyl or aryl having from 1 to 20 carbon atoms.

The perfluorinated alkyl groups are preferably selected from CF₃ or CₙF₂ₙ₊₁, wherein n is an integer between 1 and 10.

The perfluorinated alkyl group is acting as a strongly electron withdrawing group. This property plays two important roles:

1) it decreases the basicity of alcohol OH group and therefore inhibits the formation of aggregates around O⁻,

2) it increases the electrophility of the metal and thus its activity in the polymerisation of olefins.

Preferably both R² are the same and are alkyl or phenyl unsubstituted or substituted. The substituents, if present, can be selected from H, F, CF₃ or isopropyl. If the substituents are H, F or CF₃ they are located preferably at positions 2 and/or 3 and/or 4 and/or 5 and/or 6. If they are isopropyl, they preferably occupy positions 2 and 6.

Preferably R³ and R⁴ are the same and are CF₃ or CₙF₂ₙ₊₂, more preferably CF₃.

Preferably R⁵ and R⁶ are the same and are hydrogen.

The invention also discloses a metallic complex obtained by metallation of the fluorinated dialkoxy-diimine pro-ligand with a metal salt or precursor of formula MX₄ wherein M is a metal group 4 of the Periodic Table,

wherein each X is the same or different and is halogen, preferably chlorine, or alkyl or benzyl.

Preferably M is selected from Ti and Zr.

The solvent may be selected from dichloromethane (DCM) or tetrahydrofuran (THF) or a hydrocarbon such as toluene and the complexation reaction is carried out at a temperature of from 20° C. to reflux.

The amount of ligand is of one equivalent of ligand per metallic equivalent.

The present invention further discloses an active catalyst system comprising the metallic complex and an activating agent having an ionising action.

It can be aluminoxane and comprise oligomeric linear and/or cyclic alkyl aluminoxanes represented by formula

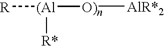

for oligomeric, linear aluminoxanes and by formula

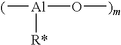

for oligomeric, cyclic aluminoxane,

wherein n is 1-40, preferably 10-20, m is 3-40, preferably 3-20 and R* is a C₁-C₈ alkyl group and preferably methyl.

The amount of activating is selected to give an Al/M ratio of from 100 to 3000, preferably of about 1000.

Suitable boron-containing activating agents may comprise a triphenylcarbenium boronate such as tetrakis-pentafluorophenyl-borato-triphenylcarbenium as described in EP-A-0427696, or those of the general formula [L′-H]+[B Ar₁ Ar₂ X₃ X₄]— as described in EP-A-0277004 (page 6, line 30 to page 7, line 7). The amount of boron-containing activating agent is selected to give B/M ratio of from 0.5 to 5, preferably of about 1.

In another embodiment, according to the present invention, the metallic complex may be deposited on a conventional support impregnated with an activating agent. Preferably, the conventional support is silica impregnated with methylaluminoxane (MAO).

Alternatively, it can be an activating support such as fluorinated alumina silica.

The present invention further discloses a method for preparing an active catalyst system that comprises the steps of:

a) providing a fluorinated dialcohol-diimine pro-ligand;

b) complexing the pro-ligand of step a) with a metallic salt or precursor MX₄ in a solvent;

c) retrieving a catalyst component;

d) activating the catalyst component with an activating agent having an ionising action;

e) optionally adding a cocatalyst;

f) retrieving an active oligomerisation or polymerisation catalyst system.

Alternatively, in step d), the catalyst component is deposited on a support impregnated with an activating agent or on a fluorinated activating support.

The cocatalyst may be selected from triethylaluminium, triisobutylaluminum, tris-n-octylaluminium, tetraisobutyldialuminoxane or diethyl zinc.

The active catalyst system is used in the oligomerisation and in the homo- or co-polymerisation of ethylene and alpha-olefins.

The present invention discloses a method for the oligomerisation or the homo- or co-polymerisation of ethylene and alpha-olefins that comprises the steps of:

a) injecting the active catalyst system into the reactor;

b) injecting the monomer and optional comonomer;

c) maintaining under polymerisation conditions;

d) retrieving the oligomers and/or polymer.

The pressure in the reactor can vary from 0.5 to 50 bars, preferably from 5 to 25 bars.

The polymerisation temperature can range from 10 to 100° C., preferably from 50 to 85° C.

Preferably the monomer and optional comonomer are selected from ethylene, propylene, 1-hexene, 1-octene or styrene.

The present invention also discloses the polymers and oligomer compositions obtained with the new catalyst systems.

### LIST OF FIGURES

FIG. 1 represents the crystal structure of pro-ligand 3.

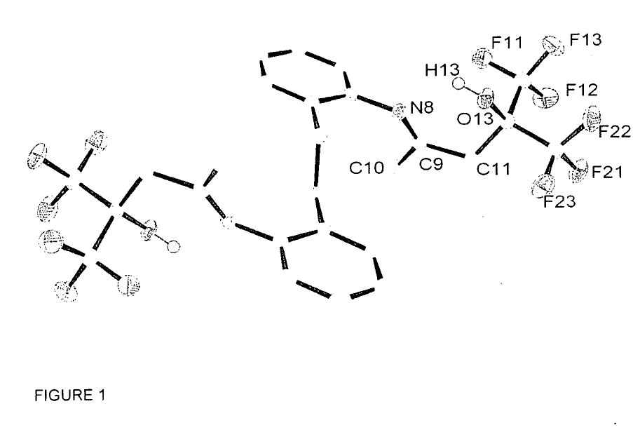

FIG. 2 represents the crystal structure of complex 5.

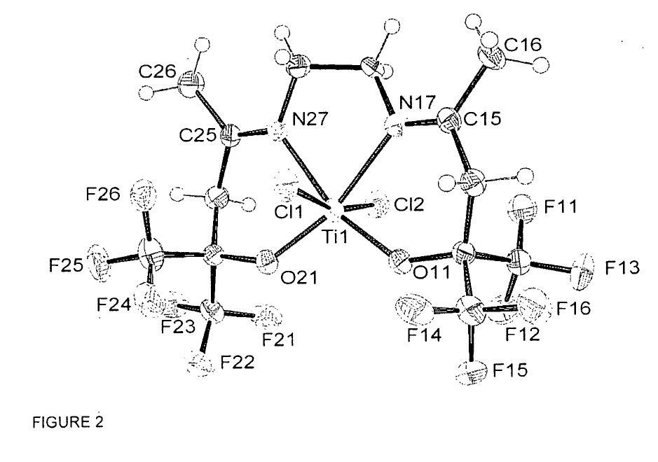

### EXAMPLES

Synthesis of 1,1,1-trifluoro-4-[(2-{[4,4,4-trifluoro-3-hydroxy-1-methyl-3-(trifluoromethyl)butylidene]amino}ethyl)imino]-2-(trifluoromethyl)pentan-2-ol (1)

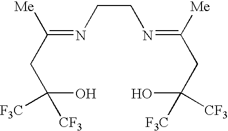

A Schlenk tube was charged with 0.80 g of montmorillonite, 10 mL of chloroform and 2.75 g (12.27 mmol) of 4,4,4-trifluoro-3-hydroxy-3-(trifluoromethyl)butan-2-one. Under an argon atmosphere, 0.36 g (6.13 mmol) of 1,2- ethylene-diamine (0.36 g, 6.13 mmol) were added and the reaction mixture was refluxed for a period of time of 72 h. The reaction mixture was filtered off and the filtrate was concentrated under reduced pressure. The resulting oily product was recrystallised from methanol at room temperature to give 3.88 g of 1 as an orange solid with a yield of 69%.

NMR results for 1 were as follows:

¹H NMR (500 MHz, CDCl₃, 298K): δ (ppm) 2.04 (s, 6H, Me), 2.73 (s, 4H, CH₂), 3.66 (s, 4H, CH₂N), 9.88 (s, 2H, OH). ¹³C NMR (75 MHz, CDCl₃, 298K): δ (ppm) 20.63 (CH₂), 33.41 (Me), 50.42 (CH₂—N), 121.1 (C—OH), 125.5 (C—F), 171.42 (C=N). ¹⁹F NMR (182 MHz, CDCl₃, 298K): δ(ppm) −78.9 (s, 12 F).

Anal. Calcd. for C₁₄H¹⁶F₁₂N₂O₂: C, 35.60; H, 3.41. Found: C, 36.44; H, 3.89.
Synthesis of Racemic 4,4′-(cyclohexane-1,2-diylbis(azan-1-yl-1-ylidene))bis(1,1,1-trifluoro-2-(trifluoromethyl)pentan-2-ol) (rac-trans-2)

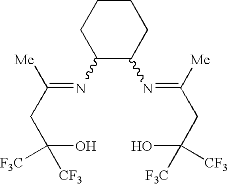

pro-Ligand 2 was prepared using the same procedure as that described for pro-ligand 1 starting from 0.80 g of montmorillonite, 2.50 g (11.1 mmol) of fluorinated aldol 4,4,4-trifluoro-3-hydroxy-3-(trifluoromethyl)butan-2-one and 0.63 g (5.5 mmol) of trans-1,2-diaminocyclohexane to give 3.97 g of 2 as a white solid with a yield of 68%.

The melting temperature was of 108.4° C.

NMR results for rac-2 were as follows:

¹H NMR (500 MHz, CDCl₃, 298K): δ (ppm) 1.38-1.84 (m, 8H, cyclohexyl), 2.01 (s, 6H, Me), 2.65 (m, 2H, CH—N du cyclohexyl), 10.19 (s, 2H, OH).

¹³C NMR (75 MHz, CDCl₃, 298K): δ (ppm) 170.0 (2C, CN), 21.10 (2C, CH₂—C—CF₃), 24.20 (2C, CH₃), 32.02 (2C, CH₂ from cyclohexyl), 39.10 (2C, CH₂—CN from cyclohexyl), 64.30 (2C, CH—N de cyclohexyl), 122.0 (2C, CO), 126.0 (2C, q, J_{CF}=CF₃).

¹⁹F NMR (182 MHz, CDCl₃ 298K): δ (ppm) −78.72 (q, J=10.5 Hz, 6F), −79.20 (q, J=10.34 Hz, 6F).

HR-MS (70 eV, El): m/z calc. for C₁₈H₂₂F₁₂N₂O₂: 526.1490; found: 526.1521 (6 ppm). [M—CF₃]⁺ (C₁₇H₂₂N₂O₂F₉); theor. mol. weigh: 457.1538; found: 457.1568.
Synthesis of 4,4′-((1R,2R)-cyclohexane-1,2-diylbis(azan-1-yl-1-ylidene))bis(1,1,1-trifluoro-2-(trifluoromethyl)pentan-2-ol) (R,R-2)

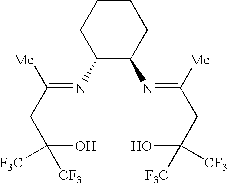

(R,R)-diol 2 was prepared using a procedure similar to that used to prepare racemic diol 2, starting from 1.20 g of montmorillonite, 4.24 g (18.95 mmol) of fluorinated aldol 4,4,4-trifluoro-3-hydroxy-3-(trifluoromethyl)butan-2-one and 0.63 g (5.5 mmol) of (1R,2R)-diaminocyclohexane to give 6.97 g of (R,R2 with a yield of 70%.

NMR results for (R,R)-2 were as follows:

¹H NMR (200 MHz, CDCl₃, 298K): δ (ppm) 1.42-1.85 (m, 8H, cyclohexyl), 2.02 (s, 6H, 2Me), 2.66 (s, 4H, cyclohexyl), 3.57 (m, 2H, CH—N), 10.19 (s, 2H, OH). ¹⁹F NMR (182 MHz, CDCl₃, 298K): δ (ppm) −78.74 (q, J=10.35 Hz, 6F), −79.22 (q, J=10.35 Hz, 6F).
Synthesis of 4,4′-(2,2′-(ethane-1,2-diyl)bis(2,1-phenylene))bis(azan-1-yl-1-ylidene)bis(1,1,1-trifluoro-2-trifluoromethyl)pentan-2-ol) (3)

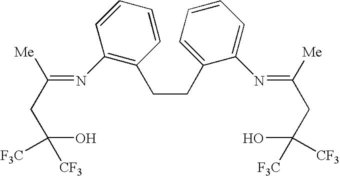

In a 50 mL flask, a mixture of 3.00 g (13.39 mmol) of fluorinated aldol 4,4,4-trifluoro-3-hydroxy-3-(trifluoromethyl)butan-2-one and 0.95 g (4.48 mmol) of 2-[2-(2-aminophenyl)ethyl]phenylamine in 40 mL of toluene, in the presence of a catalytic amount of paratoluenesulfonic acid (PTSA), was refluxed for 100 h using a Dean-Stark apparatus. The reaction mixture was filtered off, the filtrate was evaporated in vacuum and the residue was recrystallised from a CH₂Cl₂/toluene mixture to give 1.10 g of 3 as colourless crystals with a yield of 39%.

NMR results for 3 were as follows:

¹H NMR (200 MHz, CD₂Cl₂, 298K): δ (ppm) 1.95 (s, 6H, CH₃), 2.70 (s, 4H, CH₂), 2.97 (s, 4H, CH₂), 6.62 (m, 2H, arom.), 7.19 (m, 6H, arom.), 9.35 (s, 2H, OH).

¹⁹F NMR (188 MHz, CD₂Cl₂, 298K): δ (ppm) −78.9.

Anal. Calcd. for C₂₆H₂₄F₁₂N₂O₂: C, 50.01; H, 3.87. Found: C, 51.00; H, 4.28.
Synthesis of [OC(CF₃)₂CH₂C(CH₃)═NCH₂CH₂N═C(CH₃)CH₂C(CF₃)₂O]Zr(CH₂Ph)₂(4)

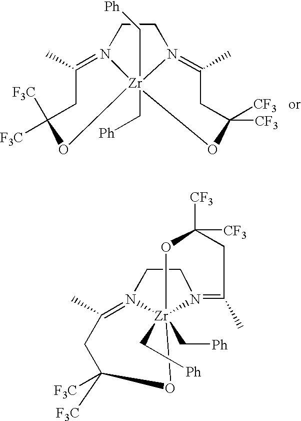

1) NMR-scale synthesis: A Teflon-valved NMR tube was charged with 25.7 mg (54.42 μmol) of diol 1 and 24.8 mg (54.42 _{μmol) of Zr(CH}₂Ph)₄. About 0.5 mL of dry toluene-d₈ were added under vacuum. The tube was kept for a period of time of 3 to 4 hours at a temperature of −30° C. and NMR spectra were recorded. The formation of 4 proceeded with a yield of about 95%, as determined by ¹H NMR.

2) Preparative synthesis: to a solution of 0.42 g (0.86 mmol) of fluorinated aldol 4,4,4-trifluoro-3-hydroxy-3-(trifluoromethyl)butan-2-one in 4 mL of toluene, was added a solution of 0.41 g (0.89 mmol) of Zr(CH₂Ph)₄ in 3 mL of toluene at a temperature of −30° C. under vigorous stirring. The reaction mixture was kept at a temperature of −30° C. overnight, after which 4 precipitated as a yellow crystalline solid. The product was separated and dried under vacuum to give 0.25 g of 4 with a yield of 38%.

NMR results for 4 were as follows:

¹H NMR (500 MHz, CD₂Cl₂, 298K): δ (ppm) 2.13 (s, 4H, ZrCH₂Ph), 2.16 (s, 6H, CH₃), 2.89 (s, 4H, OC(CF₃)₂CH₂), 3.66 (s, 4H, NCH₂), 6.68 (d, ³J=7.5 Hz, 4H, o-Ph), 6.75 (t, ³J=7.5 Hz, 2H, p-Ph), 7.04 (m, ³J=7.5 Hz, 4H, m-Ph).

¹³C{¹H} NMR (125 MHz, CD₂Cl₂, 298K): δ (ppm) 21.1 (CH₂Ph), 24.0 (CH₃), 40.6 ((CF₃)2CH₂), 51.3 (NCH₂), 79.5 (C(CF₃)₂), 119.9 (p-Ph), 123.8 (C(CF₃)₂), 125.5 (o-Ph), 127.6 (m-Ph), 151.1 (i-Ph), 177.5 (N═C(CH₃)).

¹⁹F NMR (188 MHz, CD₂Cl₂, 298K): δ (ppm) −76.9. Anal. Calcd. for C₂₈H₂₈F₁₂N₂O₂Zr:C, 45.22; H, 3.79. Found: C, 45.77; H, 3.84.
Synthesis of [OC(CF₃)₂CH²C(CH₃)═NCH₂CH₂N═C(CH₃)CH₂C(CF₃)₂O]TiCl₂ (5)

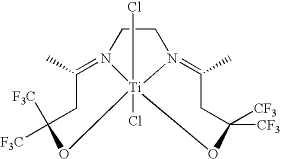

To a solution of 100 mg (0.21 mmol) of diol 1 in 4 mL of toluene, was added a solution of 20.1 mg (0.105 mmol) of TiCl₄ and 30.1 mg (0.105 mmol) of Ti(O′Pr)₄ in 4 mL of toluene at a temperature of −30° C. The reaction mixture was kept at a temperature of −30° C. overnight, and colourless crystals precipitated. The crystals were separated, washed with a minimal amount of toluene and dried under vacuum to give 70 mg of 5 with a yield of 56%.

NMR results for 5 were as follows:

¹H NMR (500 MHz, CD₂Cl₂, 298K): δ (ppm) 2.41 (s, 6H, CH₃), 3.66 (s, 4H, C(CF₃)₂CH₂), 4.20 (s, 4H, NCH₂). ¹³C{¹H} NMR (125 MHz, CD₂Cl₂, 298K): δ (ppm) 23.6 (CH₃), 41.8 ((CF₃)₂CH₂), 46.7 (NCH₂), 85.3 (C(CF₃)₂), 122.2 (C(CF₃)₂), 173.9 (N═C(CH₃)).

¹⁹F NMR (188 MHz, CD₂Cl₂, 298K): δ (ppm) −76.2.

Anal. Calcd. for C₁₄H₁₄Cl₂F₁₂N₂O₂Ti: C, 28.55; H, 2.40. Found: C, 28.96; H, 2.59.

Polymerisation of ethylene and styrene.

The polymerisations was carried out in 50 mL of toluene, at room temperature and for a period of time of 30 minutes.

12 mg of catalyst component

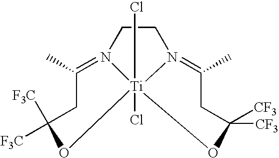

were used with 500 equivalents of methylaluminoxane (MAO) and under an ethylene pressure of 1 bar. No polymers were formed.

The same catalyst component was used for the polymerisation of styrene with 500 equivalents of MAO and 1000 equivalents of styrene and for a period of time of 2 hours.

At room temperature the activity was of 28 kg/mol/h and at a temperature of 50° C., it was of 7 kg/mol/h.

1-12. (canceled)

13. A metallic complex formed by a process comprising:
reacting via a metallation reaction, a metal salt or precursor of formula MX₄, wherein M is a metal group 4 of the Periodic Table, and wherein each X is the same or different and is a halogen, an alkyl or a benzyl group with a fluorinated dialcohol-diimine pro-ligand of general formula 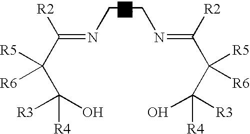
wherein R′ is a bridge having from 1 to 6 carbon atoms, which is optionally substituted so that the substituents form a ring either with each other or with R²;
each R² is independently selected from hydrogen, C₁ to C₂₀ aryl groups or C₁ to _{C}₂₀ alkyl groups optionally substituted with one or more fluorinated groups;
R³ and R⁴ are each independently selected from CF₃, CₙF₂ₙ₊₁, wherein n is an integer between 1 and 10, C₁ to C₂₀ aryl groups or C₁ to C₂₀ alkyl groups; and
R⁵ and R⁶ are each independently selected from hydrogen, C₁ to C₂₀ aryl groups or C₁ to C₂₀ alkyl groups.

14. The metallic complex of claim 13, wherein at least one of R³ and R⁴ is a perfluorinated alkyl group.

15. The metallic complex of claim 13, wherein both R² are the same and are phenyl groups.

16. The metallic complex of claim 13, wherein both R² are phenyl groups substituted with H, F or CF₃ at positions 2, 3 or combinations thereof, 4, 5, 6 with isopropyl at positions 2 and 6.

17. The metallic complex of claim 13, wherein R³ and R⁴ are the same and are CF₃ or CnF₂ₙ₊₁.

18. The metallic complex of claim 13, wherein R⁵ and R⁶ are the same and are hydrogen.

19. The metallic complex of claim 13, wherein X is selected from chlorine, methyl and benzyl.

20. A method for forming the metallic complex of claim 13 comprising:
providing the fluorinated dialcohol-diimine pro-ligand; and
complexing the fluorinated dialcohol-diimine pro-ligand with the metal salt or precursor of formula MX₄.

21. An active catalyst system comprising the metallic complex of claim 13 and an activating agent having an ionising action and optionally a cocatalyst.

22. The active catalyst of claim 21, wherein the activating agent is aluminoxane or an activating support.
